(19) United States
(12) Patent Application Publication
(10) Pub. No.: US 2014/0107574 A1
(43) Pub. Date: Apr. 17, 2014

(54) CATHETER BALLOON COATED WITH RAPAMYCIN AND SHELLAC

(71) Applicant: Rembert Pogge Von Strandmann, Bonn (DE)

(72) Inventor: Rembert Pogge Von Strandmann, Bonn (DE)

(73) Assignee: EUROCOR GMBH, Bonn (DE)

(21) Appl. No.: 14/113,953

(22) PCT Filed: Apr. 26, 2012
(86) PCT No.: PCT/EP2012/057698

§ 371 Date: Dec. 9, 2013

(30) Foreign Application Priority Data
Apr. 26, 2011 (DE) 10 2011 100 122.4

(51) Int. Cl. A61L 29/16; A61L 29/08

(52) U.S. Cl. CPC A61L 29/16; A61L 29/085
USPC 604/103.02; 427/2.25

(57) ABSTRACT

The present invention relates to a catheter balloon with a coating containing rapamycin and shellac and to a method for coating catheter balloons preferably textured catheter balloons with the pharmacological agent rapamycin, shellac and optionally further components. Moreover, the present invention relates also to the use of catheter balloons coated in such a way for the release of the pharmaceutically active agent rapamycin for prophylaxis and treatment of restenosis, preferably restenosis caused by angioplasty. The coated catheter balloons can be used alone or in combination with a coated or uncoated stent, which is crimped on the catheter balloon before or after the coating with shellac and rapamycin.

The present invention relates to a catheter balloon with a coating containing rapamycin and shellac and to a method for coating catheter balloons, preferably textured catheter balloons, with the pharmacological agent rapamycin, shellac and optionally further components. Moreover the present invention relates to the use of catheter balloons coated in such a way for the release of the pharmaceutically active agent rapamycin for prophylaxis and treatment of restenosis, preferably restenosis caused by angioplasty. The coated catheter balloons can be used alone or in combination with a coated or uncoated stent, which is crimped on the catheter balloon before or after the coating with shellac and rapamycin.

Nowadays, implantation of vessel grafts such as stents has become a well-established surgical intervention for the treatment of stenoses. In this context, the so-called restenosis (recurrent stenosis), i.e. the reocclusion of the vessel, is a frequently occurring complication. There is no exact definition of the term restenosis to be found in the literature. The most frequently used morphological definition of restenosis defines a restenosis as a reduction of the vessel diameter to less than 50% of the normal value subsequent to a successful PTA (percutaneous transluminal angioplasty). Said definition describes an empirically determined value and its hemodynamic meaning and association with clinical symptoms lack scientific background. In practice, the clinical deterioration of a patient is often considered as a sign for the occurrence of a restenosis in the previously treated vessel section.

Restenosis following stent implantation is one of the major causes for further hospitalization. Vessel traumas induced by stent implantation cause inflammatory reactions, which play a decisive role in the healing process during the first seven days. In the recent past, it has also been found that stents provided with a drug-eluting coating may cause late thromboses, i.e. in addition to restenosis the stent may also lead to a long-term problem such as late thromboses.

Concerns have been raised that the biostable or bioreabsorbable polymeric matrix of the stent, in which the drug is embedded, might induce a sustained inflammation with an increased neointimal proliferation. Additionally, the reached active agent concentration in the tissue is not homogenous: it is highest near to the stent struts, and lowest between the stent struts; this causes a non-uniform inhibition of smooth muscle cell proliferation and may induce a delayed and in-homogenous reendothelialization in different stent segments. For both mechanisms a significant contribution to late thromboses and in-stent restenosis has been discussed. The problem of late thromboses caused by drug eluting stents like paclitaxel or rapamycin eluting stents have been described as a serious problem, which can cause the death of a patient. In comparison to active agent eluting stents, which release the active agent over a certain period of time, active agent coated catheter balloons need to immediately release the active agent, since the dilatation of a catheter balloon cannot take longer than 60 seconds in order to avoid any harm to the patient and might be repeated for two or three times. However, even repeating the dilatation in order to obtain three or four or five minutes of over-all dilatation time is still a short time release of the active agent in comparison with stents, which release the active agent over days, weeks or months.

To avoid such problems, a so-called “biological stenting” may be performed using only a coated catheter balloon without stent. Here, the vessels are dilated at a constricted site by the dilatation of a coated catheter balloon, wherein the catheter balloon is dilated for a short period of time and a sufficient amount of the pharmacological agent is transferred to the vessel wall to avoid re-constriction or reocclusion of the vessel due to the dilatation of the vessel.

Such coated catheter balloons are already known from WO 2005/089855 A1 and the international patent application WO 2004/028582 A1 discloses folded balloons which are coated, especially within the folds, with a composition of a pharmacological agent and a contrast medium. A method for spray coating catheter balloons is described in WO 2004/006976 A1.

WO 2008/046641 discloses coated implants with regard to stents, preferably showing in vitro release kinetics of stents coated with 1.0% rapamycin without shellac mixture and 1.0% rapamycin/0.5% shellac mixture. Shellac has a profound impact on stent-based rapamycin by protracting active agent release long-term. Uncoated stents with Rapamycin released the active agent more efficiently in contrast to shellac and rapamycin coated stents, which released the active agent much slower. Shellac was deemed to be useful to modulate the release kinetics of an implant-based, e.g. of a stent-based compound to slow the release kinetic (more than 60 days) that is required to prevent in-stent restenosis at 6-9 months follow up.

Such a retardation of the active agent release is actually not favourable for a catheter balloon. In contrast to a stent, with a catheter balloon it should be released as much of the coated active agent in a time frame as short as possible to shorten the inflation time to an absolute minimum. Thus, it is surprising and unexpected that a catheter balloon coated with rapamycin and shellac results in an improvement of active agent release compared to a catheter balloon that was not coated with shellac. WO 2008/046641 rather implies that shellac combined with an active agent leads to a stable formulation that releases the active agent very slowly over a long time period (months) and would not be suitable for a fast active agent release using catheter balloons.

Bruno Scheller and Ulrich Speck et al., Circulation 2004, 110, 810-814 demonstrated that catheter balloons coated with pure active agent did not show any therapeutic effect. A therapeutic effect was only achieved when Paclitaxel was combined with the contrast medium solution ULTRAVIST®. ULTRAVIST® is a solution of the contrast agent iopromide.

Due to the fact that the active agent rapamycin has proven to be particularly useful in the prevention of restenosis, while coated stents, however, are disadvantageous with respect to the late thromboses described above, it is an objective of the present invention to apply the active agent onto a catheter balloon in such manner that a coating is created, which is easily detached from the balloon and can be effectively transferred to the vessel wall so that a therapeutic effect concerning the reduction of restenosis can be achieved.

Said objective is solved by the technical teaching of the independent claims. Further advantageous embodiments of the invention result from the dependent claims, the description, the figures and the examples.

Surprisingly, it has been found that a coating method of the following type is especially suited for resolving said objective.

Said method for loading or coating of a dilatable catheter balloon comprises the following steps:
I) providing an uncoated catheter balloon;
   and
IIA) providing a solution of rapamycin and shellac;
   or
IIB) providing a solution of rapamycin and providing a solution of shellac;
   and
IIIA) coating the surface of the catheter balloon with the solution of rapamycin and shellac;
   or
IIIB) coating the surface of the catheter balloon with the solution of rapamycin and subsequently with the solution of shellac or coating the surface of the catheter balloon with the solution of shellac and subsequently with the solution of rapamycin;
IV) drying the coated catheter balloon.

The invention is furthermore directed to a catheter balloon characterized by a coating containing rapamycin and shellac. The term “uncoated”, as used herein, refers to a catheter balloon with a smooth or structured or roughened surface without any active agent coating, i.e. the balloon surface comprises no pharmaceutically effective agents and especially no anti-proliferative, anti-angiogenic or anti-restenosis active agent and no coating containing an anti-proliferative, anti-angiogenic or anti-restenosis active agent.

It was surprisingly found that such a rapamycin-shellac-coating is therapeutically highly useful in keeping blood vessels open, in reducing the late lumen loss and in reducing restenosis. Thus, the present invention provides a catheter balloon and a balloon catheter comprising a catheter balloon coated with a combination of rapamycin and shellac, which is even after short dilatation times therapeutically highly useful in keeping blood vessels open and in reducing a restriction of the vessel lumen and a restenosis.

Compared to active agent eluting stents, in which about 85% of the stented plaque surface is not covered by struts, catheter balloons coated with rapamycin and shellac allow a homogenous distribution of an antiproliferative compound, which is administered area-wide. In addition, this uniformity of release to the vessel wall enhances the efficacy of the active agent. The active agent concentration within the vessel is highest at the time of injury by the dilatation of the vessel when the inflammatory and proliferative processes are most vigorous, too.

In one embodiment the catheter balloon coated with rapamycin and shellac is further characterized by the fact that after balloon inflation for 30s preferably >25% of the rapamycin are released from the balloon surface, more preferably >30%, more preferably >40%, further more preferably >50%, even more preferably >60% most preferably >70%. Consequently, a dilatation time for a single dilatation of ≦30 seconds is preferred. Moreover, a total dilatation time of ≦60 seconds is preferred, which means that the single dilatation of ≦30 seconds is repeated once.

In another embodiment the catheter balloon coated with rapamycin and shellac is further characterized by the fact that after balloon inflation for 30 seconds a rapamycin tissue concentration of preferably >10 μM/L, more preferably >30 μM/L, even more preferably >50 μM/L, further more preferably >80 μM/L, even more preferably >100 μM/L, further preferably >120 μM/L, most preferably >140 μM/L can be achieved in the dilated segment 45 min post-dilatation.

In a further preferred embodiment the catheter balloon coated with rapamycin and shellac is further characterized by the fact that after balloon inflation for 15 s a rapamycin tissue concentration of preferably >1 μM/L, more preferably >3 μM/L, even more preferably >5 μM/L, further more preferably >8 μM/L, even more preferably >10 μM/L, further preferably >15 μM/L, most preferably >20 μM/L can be achieved in the dilated segment 45 min post-dilatation.

In another embodiment the catheter balloon is coated with rapamycin and shellac, wherein the weight ratio of rapamycin to shellac is from 100:1 to 1:100, preferably 95:1 to 1:95, more preferably 90:1 to 1:90, more preferably 85:1 to 1:85, further preferably 80:1 to 1:80, more preferably 75:1 to 1:75, more preferably 70:1 to 1:70, more preferably 65:1 to 1:65, more preferably 60:1 to 1:60, more preferably 55:1 to 1:55, more preferably 50:1 to 1:50, more preferably 45:1 to 1:45, more preferably 40:1 to 1:40, more preferably 35:1 to 1:35, more preferably 30:1 to 1:30, more preferably 25:1 to 1:25, more preferably 20:1 to 1:20, even more preferably 15:1 to 1:15, further preferably 10:1 to 1:10 and most preferably 5:1 to 1:5.

Any commercially available dilatable catheter balloon may be used as catheter balloon in accordance with the invention. So called multifold balloons can be used, as described for example in the international patent application WO 94/23787 A1 by David H. Rammler, Labintelligence, USA; or in the international patent application by Scimed Life Sciences, Inc., USA; or the international patent application WO 2004/028582 A1 by Prof. Dr. Ulrich Speck the European Patent No. EP 0519063 B1 by Medtronic Inc., USA.

Such balloons are provided with folds or wings forming essentially closed cavities when the balloon is in its deflated state but bending outwards during dilatation and being capable of releasing substances contained in the folds or respectively of pressing said substances against the vessel wall.

Such balloons are advantageous since the substances enclosed in the folds, or the rapamycin enclosed in the folds, are protected from being detached too soon during the insertion by the catheter.

To protect the active agent rapamycin from early detachment from the catheter balloon, rapamycin may also be incorporated, deposited or embedded into a carrier substance, preferably a polymeric carrier. Shellac is the most preferred biologically degradable polymeric carrier. Regardless of the source of shellac, all kinds of shellac types obtained from various locations or from different insects were able to achieve the inventive results so that any kind or sort of shellac can be used in regard to the present invention. Thus there are no limitations concerning shellacs.

Shellac is a natural resin produced from the glandular secretion of a number of species of lac-producing insects. Lac insects belong to the order of Hemiptera, superfamily Coccoidea such as Metatachardia, Laccifer, Tachordiella, and others, however, members of two families—Lacciferidae and Tachardimidae—are particularly important for the lac production. The scale insect that is commercially reared is Kerria lacca, which is also known by such synonyms as Laccifer lacca Ker, Tachardia lacca, and Carteria lacca. Kerria lacca is an Indian scale insect, which infests branches of numerous trees in Southeast Asia, such as Butea frondos Rosch, Acacia arabica Willd and Ficus religiosa Linn. Shellac is the only commercially used natural resin of animal origin and is quite different from all other natural resins. More recently, as a new awareness about the environments and the toxicity of chemical raw-material is noticeable everywhere, shellac or shellac modified resin are gaining importance due to their interesting and unique characteristics. Broken branches are sold as stick lac. After grounding and washing with water to eliminate wood and red pigments (lac dye), seed lac is obtained. Purification of seed lac gives the more homogeneous product known as shellac. Its use in Europe began towards the end of the 16^{th} century mainly as a varnish (mostly known as “French polish”) for wooden objects, musical instruments and gilding, as a protective for vinyl disks and mural paintings, as an insulating material for earlier radios and other electrical tools and as an adhesive in the restoration of pottery.

Raw material shellac consists of 70-80% resin, 4-8% dye, 6-7% hard and high gloss finished wax, 3% water, up to 9% vegetable and animal impurities and aroma substances. Shellac resin is a complicated mixture of aliphatic (60%) and sesquiterpenoid acids (32%) and their esters. Sesquiterpenoid acids are jalaric and laccijalaric acids (structures I and II) and aliphatic acids are aleuritic (III) and butolic acid.

One possibility for the chemical description of the resin molecule is a structure model in which at least 4 molecules jalaric or laccijalaric acid and aleuritic acid are alternately connected by ester bonds.

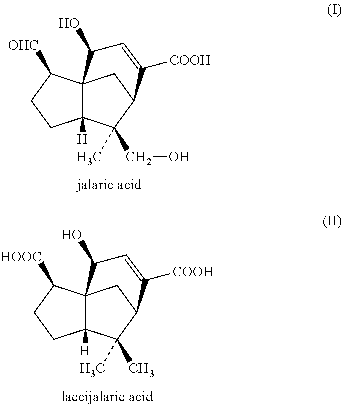

Its chemical composition is almost constant, although the amount of some components changes depending on the species of the host tree on which the insects live. By Cannizzaro-type disproportionation under alkaline hydrolysis shellolic acid (IV) and deviate compounds will be synthesized from these acids. These components are 9,10,16-trihydroxypalmitic acid (aleuritic acid) CAS [53-387-9] and shellolic acid (IV).

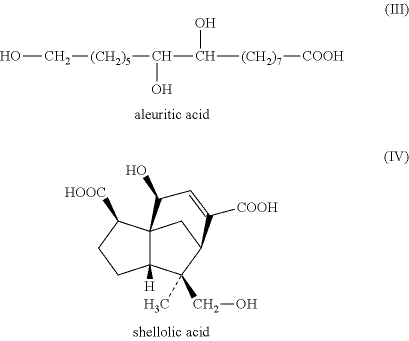

A modification with other natural or synthetic resins or co-polymerization with various monomers is possible to cross link shellac, modified shellac resins and shellac copolymers with urea, melamine, formaldehyde, isocyanides, other chemical processes such as polymerization, hydroxylation, extrication, etc. are also possible.

The following commercial grades of shellac exist:
Seedlac
Hand Made Shellac
Machine Made Shellac
Dewaxed Shellac
Dewaxed Bleached Shellac
Aleuritic Acid

Major Properties of shellac are:
Shellac is a hard natural resin
Shellac has a good resistance against solvents
Shellac is based on hydrocarbons
Shellac is non toxic
Shellac is thermoplastic
Shellac is physiologically harmless
Shellac is approved for various applications in the food industry
Shellac is not UV-resistant
Shellac is soluble in lower alcohols
Shellac has excellent dielectric properties, a high dielectric strength, a low dielectric constants, a good tracking resistance etc.
Shellac has a low melting point (65-85° C.).
Shellac is water soluble in aqueous-alkaline solutions
Coatings do not change their electric properties under UV-radiation
Shellac has excellent film forming properties
Shellac has low thermal conductivity and a low coefficient of expansion, forms smooth, high glossy films and surfaces
Shellac coating has excellent adhesion to many coatings and can be polished
Shellac can be cross linked to modify other natural/synthetic resins

Examples for industrial uses are:
Coating of pills and tablets
Coating of fruits
Cosmetics
French polish surface coating, sealer
Optical frames

The catheter balloons according to the invention were coated with different commercial grades of shellac as well as with varying batches, which differed in the Lac insects and host tree types as well as in the time of harvest. There were no differences in rapamycin release observable in the various rapamycin-shellac coated catheter balloons.

In order to apply said carrier shellac or other additional carriers onto the catheter balloon surface, the carrier substance can be added to the solution of rapamycin or can be applied as a second solution without or even again with rapamycin. Such solutions containing rapamycin and/or shellac and optionally further carrier substances are then applied onto the catheter balloon surface using conventional coating methods, in particular spattering, spraying or dipping methods. Suitable additional carriers are such substances, which are also used as balloon material, in particular polymeric and polymerizable substances as listed further below.

The rapamycin is embedded or incorporated into shellac, wherein up to 30% of the total amount being detached prematurely during the insertion of the catheter balloon, but where there is still a sufficiently high and therapeutically effective amount of rapamycin present on the balloon once it has reached its target position.

Thus, it is preferred to protect the active agent rapamycin from premature detachment by embedding into shellac on the surface of the catheter balloon and optionally underneath the folds of the balloon.

But also in such cases, where the coating, i.e. the rapamycin, is not protected by the folds of a multifold balloon or where the rapamycin is not incorporated into a larger excess of shellac, a sufficient amount of the pure active agent rapamycin can be applied onto the catheter balloon so that an effective dose is released at the target site.

Generally, an amount of 0.1 μg to 30 μg of rapamycin per mm² can be applied onto the surface of the balloon catheter to be coated, while an amount of 0.5 μg/mm² to 6 μg/mm² of rapamycin is sufficient in order to achieve the desired effect on restenosis prophylaxis. Preferably the amount of rapamycin per mm² balloon surface is between 1 μg/mm² and 5 μg/mm², more preferably between 1.5 μg/mm² and 4.5 μg/mm², still more preferably between 2.0 μg/mm² and 4.0 μg/mm², and most preferably between 2.5 μg/mm² and 3.5 μg/mm².

Preferred is also a total amount of 10 to 1000 μg of rapamycin per catheter balloon and most preferably of 20μ to 400 μg per catheter balloon.

Rapamycin is commercially available from several suppliers, like Sigma-Alderich, Merck, Selleck and Cayman. Rapamycin is known under the trademark name of Rapamune® and is also designated with various synonymous names such as: Sirolimus, Sirolimusum, D02.540.505.760, D04.345.349.760 and SRL.

Its chemical structure is as follows:

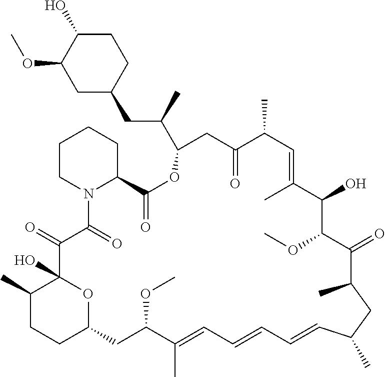 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

IUPAC nomenclature is as follows:
[(3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentone.

Rapamycin is an immunesuppresivum with macrolid structure (31 membered) and can be extracted from streptomyces Streptomyces hygroscopicus. Rapamycin inhibits a set of cytokine-mediated signaltransduction pathways by complex formation with the protein mTOR (mammalian Target of Rapamycin). This leads finally to the inhibition of the cell cycle and thus the cell division.

Rapamycin is highly soluble in dimethyl sulfoxide (DMSO) and methanol as well as in waterfree ethanol, but it is relatively bad soluble in water (2.6 μg/mL).

As solvent for rapamycin dimethyl sulfoxide (DMSO), acetone, chloroform, ethyl acetate, ethanol, and methanol are used.

The inventive coating method can be performed in two alternatives ways. A catheter balloon and preferably an uncoated catheter balloon or a catheter balloon without any releasable active agent in its surface is provided. Then, a solution of rapamycin together with shellac in a suitable solvent such as acetone, ethyl acetate, ethanol, methanol, DMSO, THF, chloroform, methylene chloride or mixtures thereof is prepared and applied using conventional coating methods such as spray coating, dip coating etc. in order to obtain after the drying step a solid rapamycin-shellac coating on the surface of the catheter balloon (steps I+IIA+IIIA+IV).

Alternatively one can prepare a rapamycin solution and a separate shellac solution and apply both solutions simultaneously or subsequently in order to obtain after the drying step a solid rapamycin shellac coating on the surface of the catheter balloon (steps I+IIB+IIIB+IV).

The coating steps IIIA) and IV) or IIIB) and IV) respectively can be repeated several times in the inventive coating methods. Usually, the coating procedure is repeated once or two or three times, but said repetition is not obligatory. Even one coating procedure may be sufficient for achieving the required amount of rapamycin and shellac on the catheter balloon.

The drying step IV) can be performed at room temperature or at elevated temperatures up to 50° C. and at atmospheric pressure or under reduced pressure up to high vacuum. If the coating step III) [IIIA) or IIIB)] is repeated, the drying steps IV) are conducted preferably at room temperature and atmospheric pressure, while preferably after the last coating step of the cycle the drying step is more intensive, i.e. longer or under vacuum or with elevated temperature.

The catheter balloon is dilatable or expandable and is most preferably an angioplasty catheter balloon, which can be used without crimped stent or with a crimped stent. As stent, all kinds of common stents, such as self-expandable stents, not self-expandable stents, metal stents, polymer stents, biodegradable stents, bifurcation stents, uncoated (bare) stents, polymer coated stents, active agent releasing stents, stents with a pure active agent coating etc. can be used.

Moreover, the stent can be crimped on the catheter balloon before the inventive coating procedure is carried out so that balloon catheter and stent are coated together with a shellac-rapamycin coating. If the catheter balloon is coated first and the stent is crimped on the balloon thereafter, a rapamycin coated stent or a rapamycin-shellac coated stent could be used having the same or different concentration of rapamycin and/or shellac on the surface.

However, it is preferred to use the coated catheter balloon of the present invention without stent.

The provided catheter balloon is normally a multifold catheter balloon which will be coated also under or within the folds. Moreover, it is possible to selectively coat or fill the folds. The coating within or under the folds has the advantage that during insertion of the catheter balloon the coating and thus the active agent rapamycin is protected against being washed off by the blood stream.

Furthermore, the catheter balloon can be coated in its expanded (inflated) or deflated state.

The preferred solvents for shellac and rapamycin are volatile, easily removable solvents such as acetone, ethyl acetate, ethanol, methanol, DMSO (dimethyl sulfoxide), THF (tetrahydrofuran), chloroform and methylene chloride.

The total catheter balloon surface loading with rapamycin and shellac is between 1 μg/mm² and 12 μg/mm². Preferably the amount of rapamycin and shellac present on the coated balloon surface is between 2 μg/mm² and 10 μg/mm² balloon surface, more preferably between 3 μg/mm² and 9 μg/mm², still more preferably between 4 μg/mm² and 8 μg/mm², still more preferably between 5 μg/mm² and 7 μg/mm², and most preferably between 5.5 μg/mm² and 6.5 rapamycin and shellac per mm² balloon surface (μg/mm²).

The inventive coating method can optionally further comprise step V):
V) Sterilization of the rapamycin and shellac coated catheter balloons.

The sterilization is most preferably performed with ethylene oxide.

Moreover, the inventive coating method can optionally further comprise step IB):
IB) Protecting the parts of the balloon catheter, which should not be coated with a removable protection cover.

Since the catheter balloon is only one part of the balloon catheter, the surfaces of the balloon catheter, which should not be coated with the rapamycin-shellac composition, can be protected by a removable protection cover such as a plastic bag or plastic foil and only the catheter balloon is left freely accessible so that only the exposed part will be coated. After the coating method is completed, the protection cover is removed.

The inventive coating method can optionally further comprise step VI):
VI) Protecting the coated catheter balloon with a removable protection cover.

The removable protection cover is useful to protect the ready coated catheter balloon and especially the coating on the catheter balloon from damage, e.g. during the transport or storage.

As described below in detail, the surface of the catheter balloon is textured, smooth, rough, harsh, provided with cavities or provided with channels open towards the outside of the balloon.

The coating solution containing rapamycin can optionally contain at least one further carrier substance alongside shellac. Said at least one further carrier substance is selected from the group consisting of or comprising:

parylene C, parylene D, parylene N, parylene F, polyvalerolactones, poly-□-decalactone, polylactonic acid, polyglycolic acid, polylactides, polyglycolides, copolymers of the polylactides and polyglycolides, poly-□-caprolactone, polyhydroxybutyric acid, polyhydroxybutyrates, polyhydroxyvalerates, polyhydroxybutyrate-co-valerate, poly(1,4-dioxane-2,3-dione), poly(1,3-dioxane-2-one), poly-para-dioxanone, polyanhydrides, polymaleic acid anhydride, polyhydroxymethacrylates, fibrin, polycyanoacrylate, polycaprolactone dimethylacrylates, poly-β-maleic acid, polycaprolactone butyl acrylates, multiblock polymers from oligocaprolactonedioles and oligodioxanonedioles, polyether ester multiblock polymers from PEG and poly(butylene terephthalate), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate), polyorthoesters, polyglycolic acid trimethyl-carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxides, propylene oxides, soft polyurethanes, polyurethanes having amino acid residues in the backbone, polyether esters, polyethylene oxide, polyalkene oxalates, polyorthoesters as well as their copolymers, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivatives thereof, heparins, chondroitin sulfate, dextran, β-cyclodextrins, copolymers with PEG and polypropylene glycol, gum arabic, guar, gelatin, collagen N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylene, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones, polysiloxanes, polydimethylsiloxanes, polyvinyl halogens and copolymers, cellulose ethers, cellulose triacetates, chitosans and copolymers and/or mixtures of the aforementioned polymers.

In the case that a textured surface of the catheter balloon is desired, the surface of the catheter balloon can be textured mechanically, chemically, electronically and/or by means of radiation to allow for an improved adhesion of rapamycin and to facilitate the precipitation or crystallization of the rapamycin.

At the texturing of the surface of the catheter balloon the surface of the catheter balloon has to be modified in the range from nanometers to micrometers, i.e. a kind of micro-rough surface structure has to be provided. Surface texturing is preferably applied to the whole area to be coated of the catheter balloon and may result in organized or random structures.

The catheter balloons may be composed of the following materials:

parylene C, parylene D, parylene N, parylene F, polyvalerolactones, poly-□-decalactone, polylactonic acid, polyglycolic acid, polylactides, polyglycolides, copolymers of the polylactides and polyglycolides, poly-□-caprolactone, polyhydroxybutyric acid, polyhydroxybutyrates, polyhydroxyvalerates, polyhydroxybutyrate-co-valerate, poly(1,4-dioxane-2-dione), poly(1,3-dioxane-2-one), poly-para-dioxanone, polyanhydrides, polymaleic acid anhydride, polyhydroxymethacrylates, fibrin, polycyanoacrylate, polycaprolactone dimethylacrylates, poly-β-maleic acid, polycaprolactone butyl acrylates, multiblock polymers from oligocaprolactonedioles and oligodioxanonedioles, polyether ester multiblock polymers from PEG and poly(butylene terephthalate), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate), polyorthoesters, polyglycolic acid trimethyl-carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxides, propylene oxides, soft polyurethanes, polyurethanes having amino acid residues in the backbone, polyether ester, polyethylene oxide, polyalkene oxalates, polyorthoesters as well as their copolymers, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivatives thereof, heparins, chondroitin sulfate, dextran, β-cyclodextrins, copolymers with PEG and polypropylene glycol, gum arabic, guar, gelatin, collagen N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylene, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones, polysiloxanes, polydimethylsiloxanes, polyvinyl halogens and copolymers, cellulose ethers, cellulose triacetates, chitosans and copolymers and/or mixtures of the aforementioned polymers.

Polyamides, block copolymers of polyamide-polyether-polyester, polyurethanes, polyester and polyolefins are preferred.

It is of importance to avoid all damage to the catheter balloons while the balloon surface is textured and to ensure that their capability to expand is not disadvantageously affected. Thus, the methods for micro texturing the balloon surface must not cause the formation of holes, micropores or fissures in the balloon material. Ideally, only the outer surface of the balloon, i.e. to a maximum depth of 1 mm, is textured.

The dilatable catheter balloon may be textured mechanically by making use of a rasp-like device, a rasp or a blasting method employing solid particles, such as a sand blasting procedure.

In a chemical-mechanical procedure a suspension or a dispersion of solid particles in a solvent, in particular in water, is used. Such methods are also referred to as chemical polishing methods. By rubbing of such compositions onto the surface of the balloon material, the material is roughened without deep fissures or holes arising.

In a purely chemical texturing method, acids, bases, etching chemicals and/or oxidizing chemicals corroding the surface of the balloon material are used. Such chemicals, however, have to be used with caution, as the balloon material could be damaged, if the exposition period is too long or the chemicals are too much concentrated.

When an electrical or electronic procedure is used for the texturing of the surface of the dilatable catheter balloon, the texturing is performed by means of conductors, which are heated by electrical current flow. For example, a fine, warm, hot or glowing needle may be used to melt the surface of the balloon material whereby certain patterns can be created on the surface, especially when the needle is moved along the surface of the catheter balloon.

An elegant method for generating organized structures, especially in form of micro depressions or micro channels, can consist of the use of lasers or basically of strongly focused radiation. Said radiation means are very accurate and may be especially used for the generation of defined textures such as grids, spirals or lines.

The textured or micro modified to nano-modified surface of the catheter balloon as well as the not textured catheter balloons may be wetted by using all common methods before applying the coating solution in order to increase adhesion of the coating to the balloon surface.

In order to apply the rapamycin-shellac solution or the rapamycin solution and the shellac solution onto the balloon surface any kind of common coating method can be used such as spray coating, brush coating, dip coating, vapour deposition, pipetting and the like.

The content of rapamycin in the rapamycin containing coating solution is between 1 μg to 1 mg rapamycin per ml solution, preferably between 10 μg to 500 μg of rapamycin per 1 ml solution, more preferably between 30 μg to 300 μg of rapamycin per 1 ml solution, and most preferably between 50 μg to 100 μg of rapamycin per 1 ml solution. For example, the solution of rapamycin in ethanol, acetone, ethyl acetate or DMSO may be applied onto the balloon surface by means of spattering, dipping, plasma deposition, brushing or spraying. While the whole surface of the catheter balloon is usually coated when the dipping method or the plasma deposition method is used, spattering, brushing and spraying may be used to coat only a part of the balloon surface.

According to the invention, the catheter balloon does not have to be completely coated. Partial coating of the catheter balloon or partial loading of certain texture elements on the surface of the catheter balloon may be sufficient. A special catheter balloon including micro-needles or micro-pores or micro-chambers is disclosed in the international patent application no. WO 02/043796 A2 issued to Scimed Life Systems, Inc., USA, wherein inflatable and textured areas are present on the balloon surface. In said embodiment, loading or inflating certain portions of the balloon surface would be sufficient to achieve the desired therapeutic success, wherein it is also possible, evidently, that the whole surface is coated.

One example, where it is desirable to coat the catheter balloon only partially is the valvuloplasty. Balloon valvuloplasty is a method, in which a narrowed heart valve is stretched open using a method that does not require open heart surgery. Balloon valvuloplasty is performed to improve valve function and blood flow by enlarging the valve opening. It is a treatment for aortic, mitral, and pulmonary stenosis. In balloon valvuloplasty, a thin catheter balloon is inserted through the skin into a blood vessel in the groin area, and then threaded up to the opening of the narrowed heart valve. The balloon is inflated to stretch the valve and to relieve the valve obstruction. The prevention of restenosis is also of concern, however, catheter balloons that are coated on the whole surface are not suitable, because only a small part in the middle of the catheter balloon is in contact with the valve, wherein the rest of the catheter balloon lies in the ventricle and the atria of the heart. After inflation of the balloon, the walls in the ventricle and atria of the heart come also into contact with the catheter balloon. A release of active agent next to these healthy tissues is not desirable and could lead to severe side effects. The catheter balloon for balloon valvuloplasty according to the invention is only coated in the area that comes into direct contact with the valve, where an inhibition of restenosis is wanted. Thus, a preferred embodiment of the present invention is a catheter balloon for the balloon valvuloplasty coated with shellac and rapamycin, in which only that part of the catheter balloon is coated that comes into contact with the heart valve. Another preferred embodiment of the present invention is directed to a catheter balloon, which is completely coated with shellac but which is coated with rapamycin only around that part of the catheter balloon, which comes into contact with the heart valve.

Furthermore, another possibility consists of a partial coating of the catheter balloon, i.e. of certain sections of the catheter balloon and, successively, of additional areas until a completely coated catheter balloon is obtained, if desired.

Since the rapamycin-shellac coating is hard to characterize, the present invention relates also to rapamycin shellac coated catheter balloons obtained according to the inventive coating method disclosed herein as well as to balloon catheter and dilatation catheter comprising said rapamycin shellac coated catheter balloon.

Furthermore, another active agent may be added to the rapamycin containing solution. Said additional active agent can be selected from the following group comprising or consisting of:

abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-All, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a,4-hydroxyoxycyclo phosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, mycophenolate mofetil, β-lapachone, podophyllotoxin, podophyllic acid-2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, angiopeptin, monoclonal antibodies inhibiting muscle cell proliferation, bFGF antagonists, probucol, prostaglandins, 1-hydroxy-11-methoxycanthin-6-one, scopoletin, NO donors, pentaerythrityl tetranitrate and sydnoimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), rapamycin and its derivatives, 6-α-hydroxy-rapamycin, taxoteres, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF-1, active agents from the group of antibiotics, cefadroxil, cefazolin, cefaclor, cefoxitin, tobramycin, gentamicin, penicillins, dicloxacillin, oxacillin, sulfonamides, metronidazole, enoxaparin, heparin, hirudin, PPACK, protamine, prourokinase, streptokinase, warfarin, urokinase, vasodilators, dipyramidole, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators, halofuginone, nifedipine, tocopherol, tranilast, molsidomine, tea polyphenols, epicatechin gallate, epigallocatechin gallate, leflunomide, etanercept, sulfasalazine, dicloxacillin, tetracycline, triamcinolone, mutamycin, procainimide, retinoic acid, quinidine, disopyrimide, flecamide, propafenone, sotalol, natural and synthetically obtained steroids such as bryophyllin A, inotodiol, maquiroside A, ghalakinoside, mansonine, strebloside, hydrocortisone, betamethasone, dexamethasone, non-steroidal substances (NSAIDS) such as fenoprofen, fenoprofen, ibuprofen, indomethacin, naproxen, phenylbutazone, antiviral agents, acyclovir, ganciclovir zidovudine, clotrimazole, flucytosine, griseofulvin, ketoconazole, miconazole, nystatin, terbinafine, antiprotozoal agents, chloroquine, mefloquine, quinine, natural terpenoids, hippocaesculin, barringtogenol-C21-angelate, 14-dehydroagrostistachin, agroskerin, agrostistachin, 17-hydroxyagrostistachin, ovatodiolids, 4,7-oxycycloanisomelic acid baccharinoids B1, B2, B3 and B7, tubeimoside, bruceantinoside C, yadanziosides N and P, isodeoxyelephantopin, tomenphantopin A and B, coronarin A,B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-α-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, marchantin A, maytansin, lycoridicin, margetine, pancratistatin, liriodenine, oxoushinsunine, periplocoside A, deoxypsorospermin, psychorubin, ricin A, sanguinarine, manwu wheat acid, methylsorbifolin, chromones of spathelia, stizophyllin, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

Furthermore, the present invention relates to dilatable and expandable catheter balloons and in particular to multifold balloons for catheters coated according to the inventive method.

The catheter balloons are coated preferably besides shellac with essentially pure rapamycin. Thus, the catheter balloons carry preferably a layer consisting of an active agent in form of rapamycin incorporated into the biopolymer shellac, wherein in said layer only traces of solvents are present. Optionally another active agent and/or another carrier substance may be present in a same or different amount as rapamycin or shellac.

Due to the inventive coating method, the rapamycin-shellac composition dried on the surface of the catheter balloon has a special consistence, which is hard to characterize but seems to be essential for the transfer to the cell wall and the incorporation, especially into the smooth muscle cells.

In the case of multifold balloons, one part of the rapamycin-shellac containing coating is provided underneath the folds when the balloon is in its compressed, i.e. deflated state. Said amount is sufficient to achieve the desired therapeutic success even if the remaining uncovered balloon surface is not coated with the active agent rapamycin.

Therefore, the present invention also relates to balloon catheters comprising a catheter balloon coated according to the present invention with rapamycin and shellac and optionally with a further active agent and/or optionally with a further carrier substance or matrix substance.

Such catheters are preferably used for the treatment of constricted vessel segments, particularly of blood vessels, and for the treatment and prophylaxis of stenosis, restenosis, arteriosclerosis, and fibrotic vessel constriction.

Furthermore, catheter balloons which are coated according to the invention are suitable for the treatment and/or prophylaxis of in-stent restenoses, i.e. a reoccurring vessel constriction within an already implanted stent. In such cases the placement of an additional stent has been proven to be very problematic or even impracticable from a medical point of view. Such in-stent restenoses can be effectively treated without an additional stent by applying paclitaxel with the help of a catheter balloon coated according to the present invention or a catheter, whose balloon is coated according to the present invention.

Furthermore, the catheter balloons coated according to the invention are particularly suited for the treatment of small vessels, preferably such vessels having a vessel diameter of less than 2.25 mm.

The catheter balloons coated according to the invention are preferably used in the cardiovascular area, but the catheter balloons coated according to the invention are also suitable for the treatment of vessel constrictions of biliary tracts, esophagus, urinary tracts, pancreas, renal tracts, pulmonary tracts, trachea, of the small intestine and large intestine.

The following examples illustrate potential embodiments of the invention without limiting the scope of the invention to said precise examples.

### EXAMPLES

### Example 1a

A commercially available balloon catheter for angiogenesis is provided.

50 μg rapamycin is dissolved together with 100 μg shellac per ml acetone in acetone.

The solution of rapamycin and shellac is sprayed onto the catheter balloon and subsequently dried. This procedure is repeated three further times after drying the coated balloon surface. The drying step is performed at room temperature and atmospheric pressure.

After the final coating step the catheter balloon is sterilized with ethylene oxide. Then, the coated balloon surface is provided with a protection cover and packed.

### Example 1b

A commercially available balloon catheter for angiogenesis with expandable balloon composed of a polyamide is provided.

Rapamycin is dissolved in ethanol together with shellac at a concentration of 50 μg rapamycin and 100 μg shellac per ml of ethanol.

The solution of rapamycin and shellac in ethanol is applied onto the catheter balloon with a micropipette.

After the coating step the catheter balloon is dried under reduced pressure and sterilized with ethylene oxide. Then, the coated balloon surface is protected with a protection cover and packed for shipping or storing.

### Example 1c

A commercially available dilatation catheter with expandable balloon composed of a polyamide is provided.

Rapamycin is dissolved in ethanol together with shellac at a concentration of 50 μg rapamycin and 100 μg shellac per ml of ethanol.

The solution of rapamycin and shellac in ethanol is applied onto the catheter balloon by dipping (dip-coating) the catheter balloon in the solution for 10 seconds. This procedure is repeated twice.

After each coating step the catheter balloon is dried and after the final step additionally sterilized with ethylene oxide. Then, the coated balloon surface is protected with a protection cover and packed for shipping or storing.

### Example 2

A multifold balloon such as described, for example, in WO 2004/028582 A1, WO 94/23787 A1 or WO 03/059430 A1 is provided. The multifold balloon is provided with a total of 5 folds enclosing a cavity, when the balloon is in compressed state and bending outwards, when it is in expanded state so that the balloon in its expanded state has an essentially tube-like shape.

The multifold balloon is expanded and then its surface is roughened by means of a so called “chemical polishing” process, wherein a suspension of fine particles, preferably in the range of micrometers, is used in said process and said suspension is rubbed onto the surface of the expanded catheter balloon so that a roughened surface is created.

A solution of 80 μg rapamycin in 1.0 ml ethyl acetate and a solution of 100 μg shellac in THF is provided. The roughened expanded balloon is dipped several times into the said solution of rapamycin in ethyl acetate and dried at room temperature and atmospheric pressure after each dipping.

Then, the shellac solution in THF is filled into a pipette and applied onto the dry rapamycin coating on the balloon surface.

The total rapamycin load on the balloon surface is between 1 μg to 5 μg rapamycin per mm² coated balloon surface.

After sterilization, the balloon is provided with a protective cover intended to protect the active agent on the coated, dilatable catheter balloon during the transport and the storage, wherein the cover is removed prior to the insertion of the catheter by the cardiologist.

1. A catheter balloon characterized by a coating containing rapamycin and shellac.

2. The catheter balloon according to claim 1, wherein the weight ratio of rapamycin to shellac is between 10:1 and 1:10.

3. The catheter balloon according to claim 1, wherein the total surface loading of the catheter balloon with rapamycin and shellac is between 1 μg/mm² and 12 μg/mm² and/or wherein the total surface loading of the catheter balloon with rapamycin is between 0.5 μg/mm² and 6 μg/mm².

4. Method for coating a catheter balloon according to claim 1 comprising the following steps:
I) providing an uncoated catheter balloon; and
IIA) providing a solution of rapamycin and shellac;
or
IIB) providing a solution of rapamycin and providing a solution of shellac; and
IIIA) coating the surface of the catheter balloon with the solution of rapamycin and shellac;
or
IIIB) coating the surface of the catheter balloon with the solution of rapamycin and subsequently with the solution of shellac or coating the surface of the catheter balloon with the solution of shellac and subsequently with the solution of rapamycin; and
IV) drying the coated catheter balloon.

5. The method according to claim 4, wherein the solution of rapamycin and the solution of shellac or the solution of rapamycin and shellac are prepared in acetone, ethyl acetate, ethanol, methanol, DMSO, THF, chloroform, methylene chloride or mixtures of the afore-mentioned solvents.

6. The method according to claim 4, wherein the method further comprises step IB):
IB) Protecting the parts of the balloon catheter, which should not be coated, with a removable protection cover.

7. The method according to claim 4, wherein the method further comprises step VI):
VI) Protecting the coated catheter balloon with a removable protection cover.

8. The method according to claim 4, wherein at least one carrier substance is added to the coating solution containing rapamycin.

9. Method according to claim 8, wherein the at least one carrier substance is selected from the group consisting of:
parylene C, parylene D, parylene N, parylene F, polyvalerolactones, poly-□-decalactone, polylactonic acid, polyglycolic acid, polylactides, polyglycolides, copolymers of the polylactides and polyglycolides, poly-□-caprolactone, polyhydroxybutyric acid, polyhydroxybutyrates, polyhydroxyvalerates, polyhydroxybutyrate-co-valerate, poly(1,4-dioxane-2,3-dione), poly(1,3-dioxane-2-one), poly-para-dioxanone, polyanhydrides, polymaleic acid anhydride, polyhydroxymethacrylates, fibrin, polycyanoacrylate, polycaprolactone dimethylacrylates, poly-β-maleic acid, polycaprolactone butyl acrylates, multiblock polymers from oligocaprolactonedioles and oligodioxanonedioles, polyether ester multiblock polymers from PEG and poly(butylene terephthalate), polypivotolactones, polyglycolic acid trimethyl carbonates, polycaprolactone glycolides, poly(γ-ethyl glutamate), poly(DTH-iminocarbonate), poly(DTE-co-DT-carbonate), poly(bisphenol A-iminocarbonate), polyorthoesters, polyglycolic acid trimethyl-carbonate, polytrimethyl carbonates, polyiminocarbonates, poly(N-vinyl)-pyrrolidone, polyvinyl alcohols, polyester amides, glycolized polyesters, polyphosphoesters, polyphosphazenes, poly[p-carboxyphenoxy)propane], polyhydroxy pentanoic acid, polyanhydrides, polyethylene oxides, propylene oxides, soft polyurethanes, polyurethanes having amino acid residues in the backbone, polyether esters, polyethylene oxide, polyalkene oxalates, polyorthoesters as well as their copolymers, lipids, carrageenans, fibrinogen, starch, collagen, protein based polymers, polyamino acids, synthetic polyamino acids, zein, polyhydroxyalkanoates, pectic acid, actinic acid, carboxymethyl sulfate, albumin, hyaluronic acid, chitosan and derivatives thereof, heparan sulfates and derivatives thereof, heparins, chondroitin sulfate, dextran, β-cyclodextrins, copolymers with PEG and polypropylene glycol, gum arabic, guar, gelatin, collagen N-hydroxysuccinimide, phospholipids, polyacrylic acid, polyacrylates, polymethyl methacrylate, polybutyl methacrylate, polyacrylamide, polyacrylonitriles, polyamides, polyetheramides, polyethylene amine, polyimides, polycarbonates, polycarbourethanes, polyvinyl ketones, polyvinyl halogenides, polyvinylidene halogenides, polyvinyl ethers, polyisobutylenes, polyvinyl aromatics, polyvinyl esters, polyvinyl pyrrolidones, polyoxymethylene, polytetramethylene oxide, polyethylene, polypropylene, polytetrafluoroethylene, polyurethanes, polyether urethanes, silicone polyether urethanes, silicone polyurethanes, silicone polycarbonate urethanes, polyolefin elastomers, EPDM gums, fluorosilicones, carboxymethyl chitosans, polyaryletheretherketones, polyetheretherketones, polyethylene terephthalate, polyvalerates, carboxymethylcellulose, cellulose, rayon, rayon triacetates, cellulose nitrates, cellulose acetates, hydroxyethyl cellulose, cellulose butyrates, cellulose acetate butyrates, ethyl vinyl acetate copolymers, polysulfones, epoxy resins, ABS resins, silicones, polysiloxanes, polydimethylsiloxanes, polyvinyl halogens and copolymers, cellulose ethers, cellulose triacetates, chitosans and copolymers and/or mixtures of the aforementioned polymers.

10. Method according to claim 4, wherein an additional active agent is added to the rapamycin and/or shellac containing solution and wherein said additional active agent is selected from the group consisting of:
abciximab, acemetacin, acetylvismione B, aclarubicin, ademetionine, adriamycin, aescin, afromosone, akagerine, aldesleukin, amidorone, aminoglutethimide, amsacrine, anakinra, anastrozole, anemonin, anopterine, antimycotics, antithrombotics, apocymarin, argatroban, aristolactam-All, aristolochic acid, ascomycin, asparaginase, aspirin, atorvastatin, auranofin, azathioprine, azithromycin, baccatin, bafilomycin, basiliximab, bendamustine, benzocaine, berberine, betulin, betulinic acid, bilobol, bisparthenolidine, bleomycin, combrestatin, Boswellic acids and derivatives thereof, bruceanol A, B and C, bryophyllin A, busulfan, antithrombin, bivalirudin, cadherins, camptothecin, capecitabine, o-carbamoyl-phenoxyacetic acid, carboplatin, carmustine, celecoxib, cepharanthin, cerivastatin, CETP inhibitors, chlorambucil, chloroquine phosphate, cicutoxin, ciprofloxacin, cisplatin, cladribine, clarithromycin, colchicine, concanamycin, coumadin, C-type natriuretic peptide (CNP), cudraisoflavone A, curcumin, cyclophosphamide, ciclosporin A, cytarabine, dacarbazine, daclizumab, dactinomycin, dapsone, daunorubicin, diclofenac, 1,11-dimethoxycanthin-6-one, docetaxel, doxorubicin, daunamycin, epirubicin, epothilone A and B, erythromycin, estramustine, etoposide, everolimus, filgrastim, fluoroblastin, fluvastatin, fludarabine, fludarabine-5′-dihydrogen phosphate, fluorouracil, folimycin, fosfestrol, gemcitabine, ghalakinoside, ginkgol, ginkgolic acid, glycoside 1a,4-hydroxyoxycyclo phosphamide, idarubicin, ifosfamide, josamycin, lapachol, lomustine, lovastatin, melphalan, midecamycin, mitoxantrone, nimustine, pitavastatin, pravastatin, procarbazine, mitomycin, methotrexate, mercaptopurine, thioguanine, oxaliplatin, irinotecan, topotecan, hydroxycarbamide, miltefosine, pentostatin, pegaspargase, exemestane, letrozole, formestane, mycophenolate mofetil, β-lapachone, podophyllotoxin, podophyllic acid-2-ethyl hydrazide, molgramostim (rhuGM-CSF), peginterferon α-2b, lenograstim (r-HuG-CSF), macrogol, selectin (cytokine antagonist), cytokinin inhibitors, COX-2 inhibitor, angiopeptin, monoclonal antibodies inhibiting muscle cell proliferation, bFGF antagonists, probucol, prostaglandins, 1-hydroxy-11-methoxycanthin-6-one, scopoletin, NO donors, pentaerythrityl tetranitrate and sydnoimines, S-nitroso derivatives, tamoxifen, staurosporine, β-estradiol, α-estradiol, estriol, estrone, ethinyl estradiol, medroxyprogesterone, estradiol cypionates, estradiol benzoates, tranilast, kamebakaurin and other terpenoids used in cancer therapy, verapamil, tyrosine kinase inhibitors (tyrphostins), rapamycin and its derivatives, 6-α-hydroxy-rapamycin, taxoteres, mofebutazone, lonazolac, lidocaine, ketoprofen, mefenamic acid, piroxicam, meloxicam, penicillamine, hydroxychloroquine, sodium aurothiomalate, oxaceprol, β-sitosterol, myrtecaine, polidocanol, nonivamide, levomenthol, ellipticine, D-24851 (Calbiochem), colcemid, cytochalasin A-E, indanocine, nocodazole, bacitracin, vitronectin receptor antagonists, azelastine, guanidyl cyclase stimulator, tissue inhibitor of metal proteinase-1 and -2, free nucleic acids, nucleic acids incorporated into virus transmitters, DNA and RNA fragments, plasminogen activator inhibitor 1, plasminogen activator inhibitor 2, antisense oligonucleotides, VEGF inhibitors, IGF-1, active agents from the group of antibiotics, cefadroxil, cefazolin, cefaclor, cefoxitin, tobramycin, gentamicin, penicillins, dicloxacillin, oxacillin, sulfonamides, metronidazole, enoxaparin, heparin, hirudin, PPACK, protamine, prourokinase, streptokinase, warfarin, urokinase, vasodilators, dipyramidole, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators, halofuginone, nifedipine, tocopherol, tranilast, molsidomine, tea polyphenols, epicatechin gallate, epigallocatechin gallate, leflunomide, etanercept, sulfasalazine, tetracycline, triamcinolone, mutamycin, procainimide, retinoic acid, quinidine, disopyrimide, flecamide, propafenone, sotalol, natural and synthetically obtained steroids such as bryophyllin A, inotodiol, maquiroside A, ghalakinoside, mansonine, strebloside, hydrocortisone, betamethasone, dexamethasone, non-steroidal substances (NSAIDS), fenoprofen, ibuprofen, indomethacin, naproxen, phenylbutazone, antiviral agents, acyclovir, ganciclovir zidovudine, clotrimazole, flucytosine, griseofulvin, ketoconazole, miconazole, nystatin, terbinafine, antiprotozoal agents, chloroquine, mefloquine, quinine, natural terpenoids, hippocaesculin, barringtogenol-C21-angelate, 14-dehydroagrostistachin, agroskerin, agrostistachin, 17-hydroxyagrostistachin, ovatodiolids, 4,7-oxycycloanisomelic acid baccharinoids B1, B2, B3 and B7, tubeimoside, bruceantinoside C, yadanziosides N and P, isodeoxyelephantopin, tomenphantopin A and B, coronarin A,B C and D, ursolic acid, hyptatic acid A, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrin, taxamairin A and B, regenilol, triptolide, cymarin, hydroxyanopterine, protoanemonin, cheliburin chloride, sinococuline A and B, dihydronitidine, nitidine chloride, 12-β-hydroxypregnadien-3,20-dione, helenalin, indicine, indicine-N-oxide, lasiocarpine, inotodiol, podophyllotoxin, justicidin A and B, larreatin, malloterin, mallotochromanol, isobutyrylmallotochromanol, marchantin A, maytansin, lycoridicin, margetine, pancratistatin, liriodenine, oxoushinsunine, periplocoside A, deoxypsorospermin, psychorubin, ricin A, sanguinarine, manwu wheat acid, methylsorbifolin, chromones of spathelia, stizophyllin, dihydrousambaraensine, hydroxyusambarine, strychnopentamine, strychnophylline, usambarine, usambarensine, liriodenine, daphnoretin, lariciresinol, methoxylariciresinol, syringaresinol, sirolimus (rapamycin), somatostatin, tacrolimus, roxithromycin, troleandomycin, simvastatin, rosuvastatin, vinblastine, vincristine, vindesine, teniposide, vinorelbine, trofosfamide, treosulfan, temozolomide, thiotepa, tretinoin, spiramycin, umbelliferone, desacetylvismione A, vismione A and B, zeorin.

11. The method according to claim 6, wherein the method further comprises step VI):
VI) Protecting the coated catheter balloon with a removable protection cover.
